(19) United States
(12) Patent Application Publication
(10) Pub. No.: US 2020/0352655 A1
(43) Pub. Date: Nov. 12, 2020

(54) METHODS, DEVICES, AND SYSTEMS FOR AUGMENTED REALITY GUIDANCE OF MEDICAL DEVICES INTO SOFT TISSUE

(71) Applicant: ARUS Inc., Fayetteville, NC (US)

(72) Inventor: Benjamin Couillard Freese, Cary, NC (US)

(21) Appl. No.: 16/867,872

(22) Filed: May 6, 2020

Related U.S. Application Data
(60) Provisional application No. 62/843,615, filed on May 6, 2019.

(51) Int. Cl. A61B 34/20; G06T 11/00; G06T 7/00; A61B 8/08; A61B 8/00; A61B 90/00

(52) U.S. Cl. CPC A61B 34/20; G06T 11/00; G06T 7/0012; A61B 8/0841; A61B 8/461; A61B 8/5246; A61B 2090/3983; A61B 8/565; G06T 2200/24; G06T 2207/10132; A61B 2034/2063; A61B 2034/2074; A61B 90/39

(57) ABSTRACT

Disclosed herein are methods, devices, and systems for facilitating a practitioner in guiding an insertion of a medical instrument into soft tissue of a patient. According to one embodiment, a method implemented on a computing device includes receiving a first plurality of images from a medical imaging device and receiving a second plurality of images from an augmented reality (AR) device interface. The second plurality of images includes fiducial-based information on both the medical instrument and the medical imaging device. The method further includes (1) determining a predicted position of the medical instrument using fiducial-based localization with the fiducial-based information, (2) generating a third plurality of images illustrating the predicted position of the medical instrument within a first portion of the first plurality of images and within a first portion of the second plurality of images, and (4) transmitting the third plurality of images to the AR device interface.

### PRIORITY CLAIM

This application claims priority to U.S. Provisional Patent Application Ser. No. 62/843,615, filed on May 6, 2019, the entire content of which is incorporated herein by reference.

### TECHNICAL FIELD

The present invention relates to medical devices and imaging. More specifically; methods, devices, and systems are disclosed for facilitating a practitioner in guiding an insertion of a medical instrument into soft tissue of a human or veterinary patient.

### BACKGROUND

Accurate placement of a needle or other medical instrument to reach a specific target (such as a cyst or joint) within tissue underneath the skin surface is difficult for a practitioner. In order to accurately hit the target within the tissue, image guidance in the form of ultrasound, computed tomography (CT), magnetic resonance imaging (MRI), fluoroscopy, or the like may be used to visualize the target and the needle. Two dimensional ultrasound, using a transducer, displays a relatively thin plane (i.e. slice) of tissue beneath the skin surface. This slice is typically only about 1-2 millimeters thick. The needle and the target need to be simultaneously visualized within that 1-2 millimeter slice in order to ensure accurate placement. This is typically referred to as plane alignment. For the needle to hit the target, the practitioner must estimate the proper angle (i.e. trajectory) of the needle to penetrate the skin, and then advance the needle through the tissue along that trajectory towards the target until the needle becomes visible in the ultrasound image.

If an incorrect trajectory is followed, the practitioner must move the transducer back and forth to find the needle within the tissue or withdraw (i.e. remove) the needle and adjust to a different trajectory before re-advancing the needle toward the target. Incorrect needle trajectory increases procedure time, and can cause unnecessary pain and discomfort to the patient. This occurs frequently due to the fact that the practitioner must turn their attention away from the patient (where they are holding and manipulating the ultrasound transducer and needle) to look at a screen displaying the ultrasound image.

The practitioner must then advance the needle through the tissue while looking at the screen (not the patient) until the needle appears near the target in the ultrasound image. This leads to errors in needle placement and trajectory of advancement. Additionally, a steep needle trajectory with respect to the transducer, even if perfectly placed, substantially decreases or eliminates visualization of the needle in the ultrasound image due to properties of physics inherent in ultrasound. Furthermore, if the practitioner has one hand on the transducer and one hand on the needle, the practitioner is unable to turn and reach the ultrasound machine to hit a button which captures an image needed to document successful needle placement.

As such, better methods, devices, and systems are needed to support practitioners for an insertion of a medical instrument into soft tissue of a patient for hitting a precise target.

### SUMMARY

Disclosed herein are methods, devices, and systems for facilitating a practitioner in guiding an insertion of a medical instrument into soft tissue of a patient.

According to one embodiment, a method implemented on at least one computing device is disclosed. The method includes receiving a first plurality of images from a medical imaging device and receiving a second plurality of images from an augmented reality (AR) device interface. The second plurality of images includes fiducial-based information on both the medical instrument and the medical imaging device. The method further includes determining a predicted position of the medical instrument using fiducial-based localization with the fiducial-based information. Next, the method includes generating a third plurality of images illustrating the predicted position of the medical instrument within a first portion of the first plurality of images and within a first portion of the second plurality of images. Finally the method includes transmitting the third plurality of images to the AR device interface, wherein the third plurality of images may be configured for providing a video overlay to an AR display device associated with the AR device interface. In some embodiments, AR capability of the AR display device may include or be replaced with VR capability. In still other embodiments, the AR capability may include MR capability.

In some embodiments, the fiducial-based information may be provided by a first fiducial marker positioned on the medical instrument and a second fiducial market positioned on the medical imaging device. The medical imaging device may be an ultrasound imaging device and the first plurality of images may be B-mode images. In certain embodiments, the ultrasound imaging device may include an ultrasound research interface (URI).

In some embodiments, the medical instrument may be a syringe; and illustrating the predicted position of the medical instrument may include illustrating an extension of a needle associated with the syringe needle. Upon determining the extension of the needle is approximately within an imaging plane of a transducer of the ultrasound imaging device, the method may include providing an indicator within the third plurality of images. The indicator may be a halo indicator positioned around an illustration of the predicted position and the illustration may be derived from a portion of the first plurality of images.

In some embodiments, the practitioner may be a healthcare care provider and the patient may be a human patient. In other embodiments, the practitioner may a veterinary care provider and patient may be a veterinarian patient.

In some embodiments, the AR device interface may be a radio frequency (RF) wireless interface such as a wireless personal area network (WPAN) interface, a wireless local area network (WLAN) interface, or the like. In other embodiments, the AR device interface may be an optical wireless interface.

In some embodiments, the AR device interface may be provided by the AR display device. The AR display device may be an AR headset, an AR capable smartphone, an AR capable tablet, or the like. The AR display device may have a field-of-view greater than 50 degrees and may have a resolution greater than 45 pixels per degree. In certain embodiments, the AR display device may be positioned on a stand. In other embodiments the AR display device may be positioned on the practitioner's hear.

In some embodiments, the computing device and the AR device interface are embedded within the AR display device. In other embodiments, the computing device is embedded within the medical imaging device.

In another embodiment, a computing device includes a memory and at least one processor. The computing device is configured for a method for facilitating a practitioner in guiding an insertion of a medical instrument into soft tissue of a patient. The method includes receiving a first plurality of images from a medical imaging device and receiving a second plurality of images from an AR device interface. The second plurality of images includes fiducial-based information on both the medical instrument and the medical imaging device. The method further includes determining a predicted position of the medical instrument using fiducial-based localization with the fiducial-based information. Next, the method includes generating a third plurality of images illustrating the predicted position of the medical instrument within a first portion of the first plurality of images and within a first portion of the second plurality of images. Finally the method includes transmitting the third plurality of images to the AR device interface.

In another embodiment, a non-transitory computer readable storage medium is disclosed that includes instructions to be implemented on at least one computing device including at least one processor, the instructions when executed by the at least one processor cause the at least one computing device to perform a method. The method includes receiving a first plurality of images from a medical imaging device and receiving a second plurality of images from an AR device interface. The second plurality of images includes fiducial-based information on both the medical instrument and the medical imaging device. The method further includes determining a predicted position of the medical instrument using fiducial-based localization with the fiducial-based information. Next, the method includes generating a third plurality of images illustrating the predicted position of the medical instrument within a first portion of the first plurality of images and within a first portion of the second plurality of images. Finally the method includes transmitting the third plurality of images to the AR device interface.

In another embodiment, a system includes an imaging device, a computing device, and an AR display device. The computing device is configured for a method for facilitating a practitioner in guiding an insertion of a medical instrument into soft tissue of a patient. The computing device includes a memory and at least one processor. The method includes receiving a first plurality of images from a medical imaging device and receiving a second plurality of images from an AR device interface on the AR display device. The second plurality of images includes fiducial-based information on both the medical instrument and the medical imaging device. The method further includes determining a predicted position of the medical instrument using fiducial-based localization with the fiducial-based information. Next, the method includes generating a third plurality of images illustrating the predicted position of the medical instrument within a first portion of the first plurality of images and within a first portion of the second plurality of images. Finally the method includes transmitting the third plurality of images to the AR device interface.

In some embodiments, the computing device and the AR device interface may be embedded within the AR display device. In other embodiments, the computing device may be embedded within the medical imaging device.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

The foregoing summary, as well as the following detailed description of preferred embodiments, is better understood when read in conjunction with the appended drawings. For the purposes of illustration, there is shown in the drawings exemplary embodiments; however, the presently disclosed invention is not limited to the specific methods and instrumentalities disclosed. In the drawings:

FIG. 1 depicts a block diagram illustrating a system that includes a smartphone, augmented reality (AR) headset, and an ultrasound device in accordance with embodiments of the present disclosure.

FIG. 2 depicts a block diagram illustrating a smartphone in accordance with embodiments of the present disclosure.

FIG. 3 depicts a block diagram illustrating an AR headset in accordance with embodiments of the present disclosure.

FIG. 4 depicts a display screen image associated with the AR headset of FIG. 3 and the system of FIG. 1 in accordance with embodiments of the present disclosure.

FIG. 5 depicts a display screen image associated with the AR headset of FIG. 3 and the system of FIG. 1 in accordance with embodiments of the present disclosure.

FIG. 6 depicts a display screen image associated with the AR headset of FIG. 3 and the system of FIG. 1 in accordance with embodiments of the present disclosure.

FIG. 7 depicts a display screen image associated with the AR headset of FIG. 3 and the system of FIG. 1 in accordance with embodiments of the present disclosure.

FIG. 8 depicts a display screen image associated with the AR headset of FIG. 3 and the system of FIG. 1 in accordance with embodiments of the present disclosure.

### DETAILED DESCRIPTION

The following description and figures are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The methods, devices, and systems described herein utilize optical tracking of an ultrasound transducer and needle via unique images/designs/patterns (e.g. fiducial marker) placed on these devices (which are independent of one another), with respect to a head-mounted display containing integrated camera(s) allowing augmented visualization of combined/overlaid real-life, real-time ultrasound images, and virtual representations (i.e. needle guide/trajectory). The present invention does not require sensors (i.e. accelerometers, gyroscopes, electromagnetic) or infrared to track the positions or orientations of the transducer or needle, which are independent of one another. The present invention does not require position markers to be placed on the patient. The present invention does not require a fixed camera(s) or other position or orientation detection devices to be placed in the environment. The present invention allows the practitioner to perform a procedure with the patient, ultrasound image feed, transducer, and needle/instrument all present within the same field of view. The practitioner never has to move their head for any reason.

The subject matter disclosed herein relates to relates to medical devices and imaging. Methods, devices, and systems are disclosed for facilitating a practitioner in guiding an insertion of a medical instrument into soft tissue of a human or veterinary patient using augmented reality (AR), virtual reality (VR), and/or mixed reality (MR).

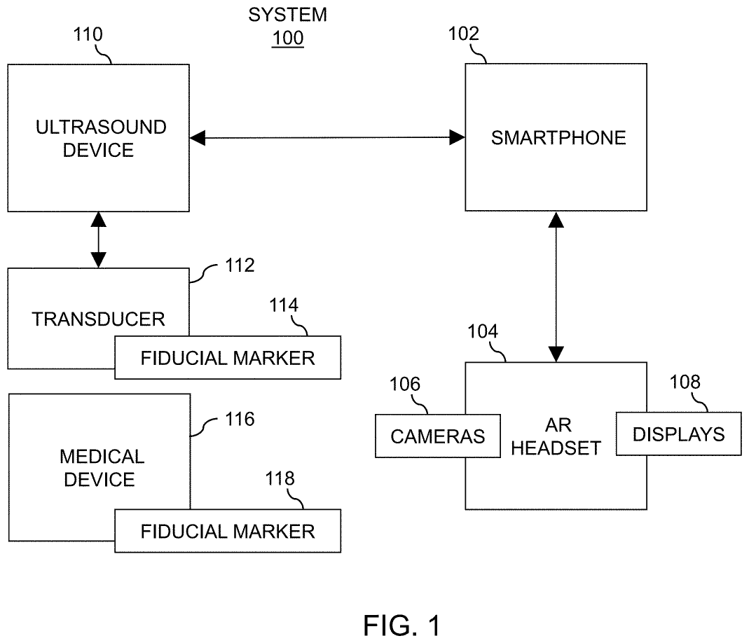

In a preferred embodiment, FIG. 1 1 illustrates a system 100 that includes a smartphone 102 (i.e. computing device). The system 100 also includes an AR headset 104 coupled with the smartphone 102 over a Bluetooth® or Wi-Fi connection. In other embodiments the AR headset 104 may be wired or optically tethered to the smartphone 102. The AR headset 104 includes cameras 106 and displays 108 for providing visual overlays for a practitioner using the AR headset 104. The system 100 also includes an ultrasound device 110 coupled with the smartphone 102 over a Bluetooth® or Wi-Fi connection. In other embodiments the ultrasound device 110 may be wired or optically tethered to the smartphone 102. In other embodiments, not shown in FIG. 1, the AR headset 104 may be replaced with an AR capable smartphone, AR capable tablet, or the like. The AR capable smartphone, AR capable table, or the like may be positioned on a fixed or mobile stand allowing hands free operation by the practitioner. In some embodiments, AR capability may include or be replaced with VR capability. In still other embodiments, the AR capability may include MR capability.

An application (not shown in FIG. 1) is configured to execute on the smartphone 102 and provide all necessary interface, processing, and communication between the ultrasound device 110 and the AR headset 104. The application may also provide additional graphical user interface (GUI) functionality for the system 100. In some embodiments, the application may be an Android® app or an iOS® app. In other embodiments, the functionality of the smartphone 102 and the application may be directly incorporated into the ultrasound device 110 and/or the AR headset 104.

The ultrasound device 110 is coupled with a transducer 112 for placement on the patient for imaging. A fiducial marker 114 is positioned on the transducer 112 allowing fiducial-based localization of the transducer 112 by the AR headset 104 and cameras 106. The system 100 also includes a medical device 116 or insertion into soft tissue of the patient by the practitioner. A fiducial marker 118 is positioned on the medical device 116 allowing fiducial-based localization of the medical device 116 by the AR headset 104 and cameras 106. In some embodiments the medical device 116 may be a syringe. The syringe may include a needle, a hub, a barrel and a plunger. The fiducial marker 118 may be positioned on the barrel of the syringe.

In other embodiments, the transducer 112 may be coupled directly to the smartphone 102 and/or the AR headset 104 via a Bluetooth, Wi-Fi, wired, or optical connection. In this embodiment, the transducer 112 is configured to send imaging data directly to the smartphone 102 and/or AR headset 104.

In this scenario, imaging data from the transducer 112 is sent to the application on the smartphone 102 where it is displayed to the practitioner via a GUI on a touch screen. The application on the smartphone 102 then communicates with a second software application on the AR headset 104 allowing the practitioner to visualize and interact with the AR content (e.g. live ultrasound feeds and needle guidance) as well as control typical ultrasound device functions via voice commands (e.g. take a picture, take a video, turn on/off Doppler, etc.) Alternatively, the smartphone 102 may be used in an AR/VR headset device (e.g. Samsung gear VR) or mounted on a stand to allow the practitioner to visualize and interact with the AR content. In this scenario a second software application designated specifically for a headset is not necessary. In another embodiment, the transducer 112 can send imaging data (e.g. Wi-Fi)) to a single software application on the AR headset 104 removing the need for the smartphone 102.

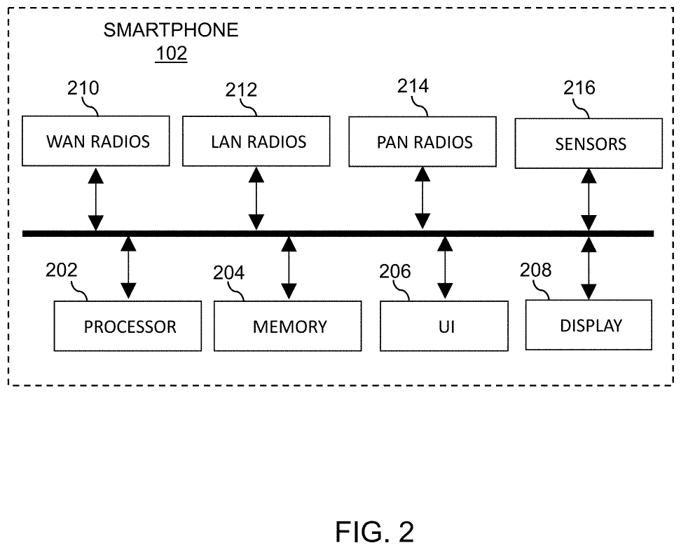

FIG. 2 illustrates a block diagram of the smartphone 102 of FIG. 1. The smartphone 102 may include at least a processor 202, a memory 204, a user interface (UI) 206, a display 208, wide area network (WAN) radios 210, local area network (LAN) radios 212, personal area network (PAN) radios 214, and sensors 216. Sensors 216 may include a global positioning system (GPS) sensor, a magnetic sensor (e.g. compass), a three-axis gyroscope sensor, an accelerometer sensor, a proximity sensor, a barometric sensor, a temperature sensor, a humidity sensor, an ambient light sensor, or the like. In some embodiments the smartphone 102 may be an iPhone® or an iPad®, using iOS® as an operation system (OS). In other embodiments, the smartphone 102 may be a mobile terminal including Android® OS, BlackBerry® OS, Windows Phone® OS, or the like.

In some embodiments, the processor 202 may be a mobile processor such as the Qualcomm® Snapdragon™ mobile processor or the like. The memory 204 may include a combination of volatile memory (e.g. random access memory) and non-volatile memory (e.g. flash memory). The memory 204 may be partially integrated with the processor 202. The UI 206 and display 208 may be integrated such as a touchpad display. The WAN radios 210 may include 2G, 3G, 4G, and/or 5G technologies. The LAN radios 1912 may include Wi-Fi technologies such as 802.11a, 802.11b/g/n, and/or 802.11ac circuitry. The PAN radios 1912 may include Bluetooth® technologies.

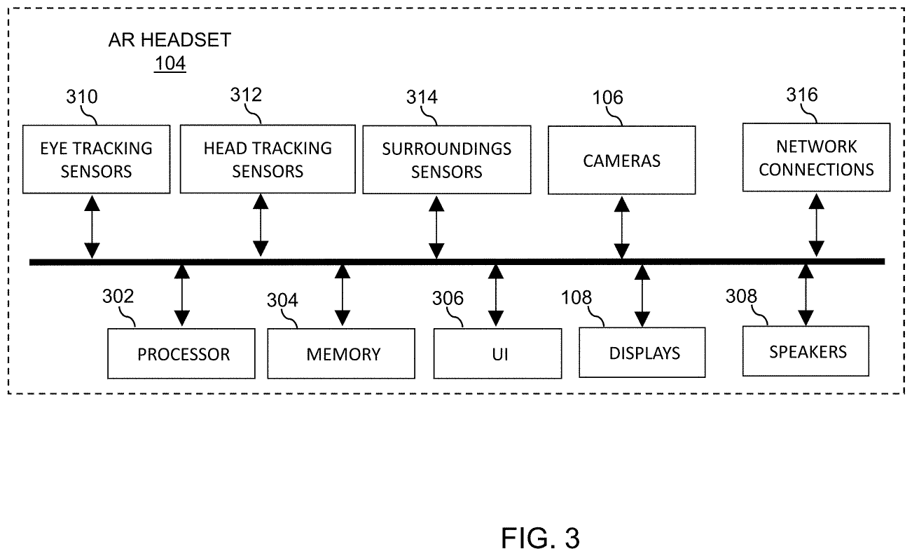

FIG. 3 illustrates a block diagram of the AR headset 104 of FIG. 1. The AR headset 104 may be an AR only headset, or may be a headset including VR, MR, and/or AR functionality. The AR headset 104 may include at least a processor 302, a memory 304, a UI 306, displays 108 (also shown in FIG. 1), and speakers 308. The memory 304 may be partially integrated with the processor 302. The memory 304 may include a combination of volatile memory (e.g. random access memory) and non-volatile memory (e.g. flash memory). The UI 306 may include a touchpad display. The displays 108 may include left and right displays for each eye of a practitioner. The speakers 108 may be positioned within the headset. In other embodiments, the speakers 308 may be provided as earbuds or headphones. Connections to the speakers 308 may be wired or wireless (e.g. Bluetooth®).

The AR headset 104 may also include eye tracking sensors 310, head tracking sensors 312, surroundings sensors 314, cameras 106, and network connections 316. The eye tracking sensors 310 may have cameras 106 co-positioned with the displays 1808. The head tracking sensors 312 may include a three-axis gyroscope sensor, an accelerometer sensor, a proximity sensor, or the like. The surroundings sensors 314 may include additional cameras positioned at a plurality of angles to view an outward circumference of the AR headset 104. The cameras 106 may include high resolutions cameras configured to provide main left eye and right eye views to the practitioner. In some embodiments, the high cameras 106 have a field-of-view greater than 50 degrees and have a resolution greater than 45 pixels per degree.

The network connections 316 may include WAN radios, LAN radios, PAN radios, or the like. The WAN radios may include 2G, 3G, 4G, and/or 5G technologies. The LAN radios may include Wi-Fi technologies such as 802.11a, 802.11b/g/n, and/or 802.11ac circuitry. The PAN radios may include Bluetooth® technologies.

In certain embodiments, the AR headset 104 may be a Microsoft® HoloLens or a Microsoft® HoloLens 2. In other embodiments, the AR headset 104 may be the Varjo Technologies XR-1 Developer Edition or the Magic Leap One. In other embodiments, the AR headset 104 may be Vuzix M300 smart glasses, a Vuzix Blade AR smart glasses, ODG R7/R8/R9 AR smart glasses, Epson Moverio BT-300/2000/2200 smart glasses, DAQRI Smart Glasses, Google Glass Enterprise Edition smart glasses, RealWear HMT-1 smart glasses, Toshiba dynaEdge™ AR smart glasses, ThirdEye X1/2 smart glasses, DreamWorld DreamGlass smart glasses, Nreal AR smart glasses, Lynx R-1 AR/VR headset, Kura Gallium glasses, or the like. In certain embodiments, the AR headset 104 may include mixed MR and VR functionality.

In other embodiments, the smartphone 102 of FIG. 1 and FIG. 2 may be replaced with a personal computer (PC), a server, a laptop, of any suitable computing device. In still other embodiments, the computing device functionality may be integrated directly into the AR headset 104 and/or the ultrasound device 110. In still other embodiments, the AR headset functionality may be directly built into a smartphone using a headset adapter to house the smartphone forming an AR headset. In other embodiments, the smartphone or a smart tablet may be mounted on a stand allowing the practitioner to view and interact with the AR content in a hands free manner.

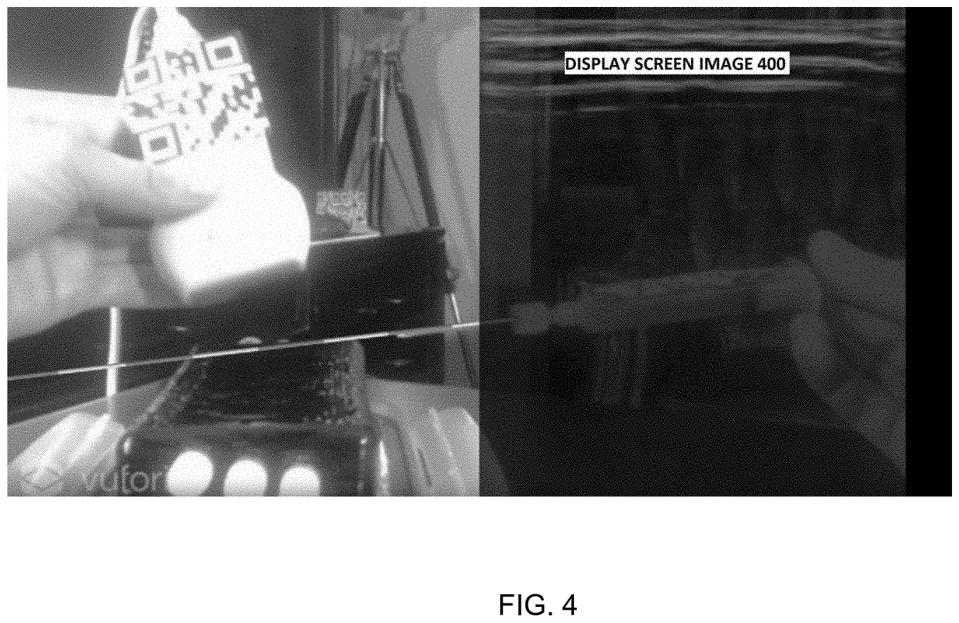

FIG. 4 depicts a display screen image 400 as seen by a practitioner via the displays 108 of system 100. The transducer 112 is shown in a left hand of the practitioner and the medical device 116 (i.e. syringe including a needle) is shown in a right hand of the practitioner. The fiducial marker 114 is shown position on the transducer 112. A silicone phantom is shown below the transducer 112. The silicon phantom is designed to simulate soft tissue of a human patient. An ultrasound image produced by the transducer 112 is shown overlaid on the right side of the display screen image 400. A predicted position (i.e. projected path or extension) of the needle is generated by the smartphone 102 and overlaid on the display screen image 400.

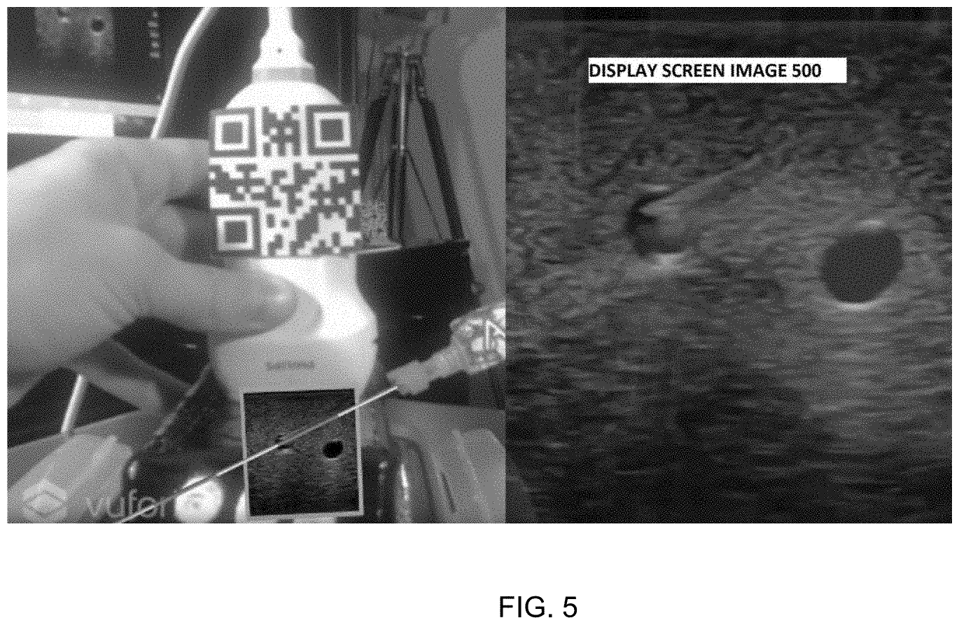

FIG. 5 depicts a display screen image 500 as seen by the practitioner via the displays 108 of system 100. Another ultrasound image of the image plane produced by the transducer 112 is shown overlaid on the right side of the display screen image 500. Upon determining the extension of the needle is approximately within the imaging, an indicator (i.e. halo and/or highlighted box) is overlaid within a smaller image version of the image plane. A portion of the fiducial marker 118 is also shown positioned on the syringe.

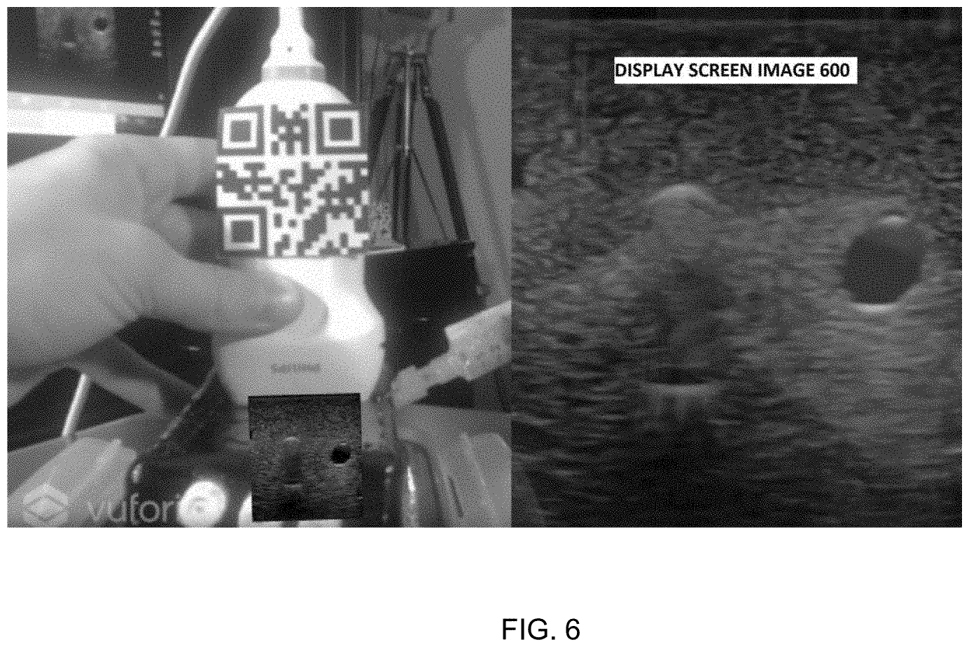

FIG. 6 depicts a display screen image 600 as seen by the practitioner via the displays 108 of system 100. As shown, the practitioner has twisted the syringe such that fiducial mark 118 is no longer viewable by cameras 106. As such the needle extension overlay is turned off allowing the practitioner to better view the actual position of the needle.

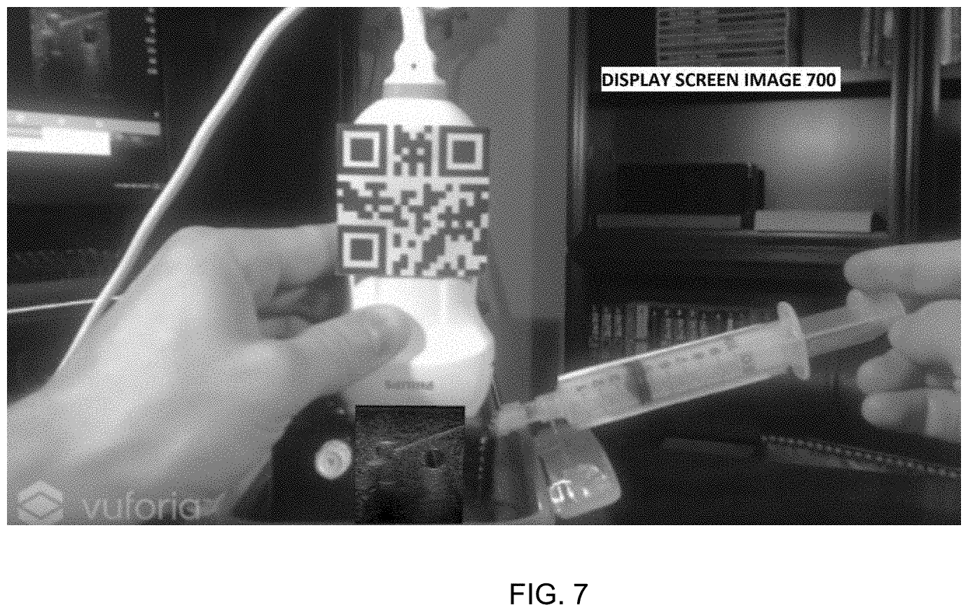

FIG. 7 depicts a display screen image 700 as seen by the practitioner via the displays 108 of system 100. In this scenario, no ultrasound image is displayed on the right allowing the practitioner more visibility of the patient, syringe, and transducer 112.

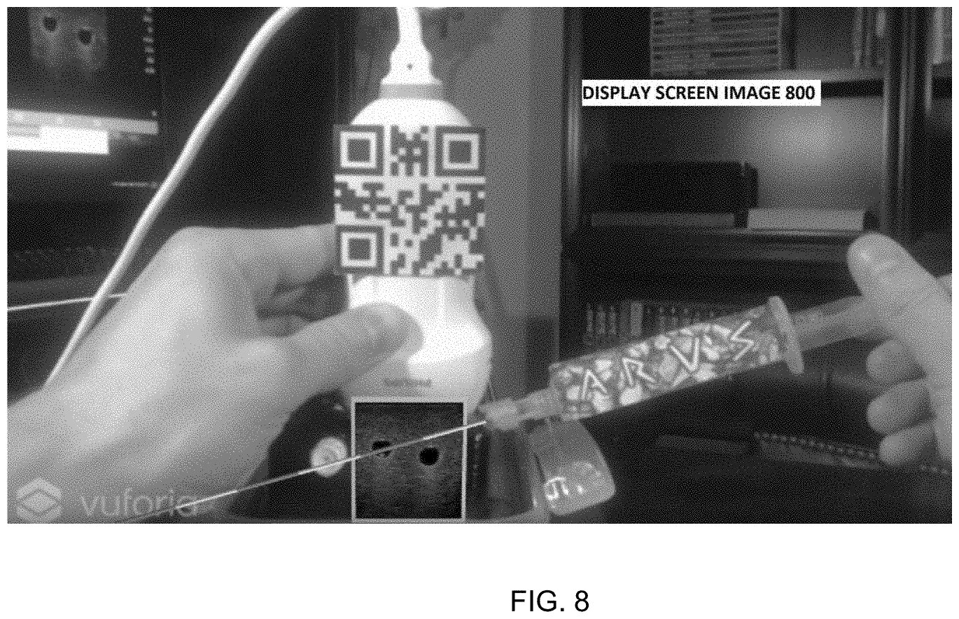

FIG. 8 depicts a display screen image 800 as seen by the practitioner via the displays 108 of system 100. The practitioner has twisted the syringe such that the fiducial marker 116 is again visible by cameras 106 and the needle extension is again overlaid onto the displays 108.

Advantages of the disclosed methods, devices, and systems include (1) virtual needle trajectory renderings on an AR overlay; (2) the ability to easily turn on/off virtual needle trajectory renderings; (3) depth and distance alignments of the transducer and the needle; (4) the practitioner does not need to look away from patient during the procedure; and (5) magnified ultrasound image in addition to a smaller image overlaid onto patient.

Additional features may include voice indication of image plane alignment with the needle and voice commands for turning the needle extension on/off. Other commands may include turning the ultrasound image on the right hand side of the display on/off. Additional commands may zoom in/out on ultrasound images. Capturing an image and/or video with the system 100 provides a significant advantage over other methods since saved images/video through the AR headset 104 replicate the practitioners exact view of the exam/procedure and capture the environment, the patient, and thus allows for third party individual(s) to validate the legitimacy of the ultrasound image/feed by its environmental context without any requisite labeling or written clarification of the actual image/video being displaying (this is the traditional method of medical documentation). It provides advanced and traditionally absent documentation of context (e.g. patient position, orientation, etc.) in addition to the traditional ultrasound systems which only save images/video of the ultrasound feed.

The following paragraphs will disclose additional features, descriptions, and embodiments of the system 100 of FIG. 1 and/or variations thereof.

Real-time independent position (X, Y, Z) and orientation (rotation) tracking of the transducer and needle is accomplished using an unrestrained or free-hand guidance system which has no environmental requirements other than an ambient light source (natural and/or artificial lighting), allowing an AR/MR/VR display device (e.g. head-mounted or stand-mounted) with at least one built-in camera to detect and recognize unique images/designs/patterns printed on or affixed to both the transducer and needle. This may also be accomplished via the emerging technology of visible light spectrum time of flight (TOF) cameras for depth sensing of the transducer and needle/instrument.

Via the head-mounted or stand-mounted AR/MR/VR display, the practitioner then is able to visualize two real-time or live ultrasound feeds: 1) a large/magnified view, such as a virtual monitor/screen which is always present in the field of vision. This large view can be displayed in multiple ways. For example, it can be visible on the top, bottom, left or right side of the AR/MR/VR field of view. 2) A true-to-life/shown to scale of actual tissue size view which is virtually “attached” to the bottom of the transducer, projecting away (into the tissue), and having correct orientation with respect to the transducer. Ultrasound feed 2 is visible to the operator as long as the integrated head-mounted display camera(s) can detect the unique image/pattern/design printed on or affixed to the transducer. Both live ultrasound feeds visible in the head-mounted display allow the practitioner to perform a free-hand diagnostic scan and/or identify a target for needle guided procedure (e.g. aspiration, injection, biopsy, ablation, etc.).

Simultaneously, a needle or other procedural instrument with a unique image/pattern/design printed on or affixed to it can be recognized by the head-mounted AR display device camera(s) at which time the practitioner visualizes the real-life needle/syringe or instrument as well as an overlaid virtual rendering or representation of the needle extending past the tip indicating linear trajectory. Using free-hand manipulation of the needle or instrument, the practitioner is able to select an angle of insertion and linear trajectory of the needle to reach the target. This is accomplished when the virtual needle trajectory rendering/representation can be seen intersecting the target in the live ultrasound feed displayed below the transducer.

Virtual indicia appear when the virtual needle trajectory rendering/representation intersects the virtual projection of the ultrasound feed below the transducer (e.g. a highlighted box and/or halo appears and surrounds the ultrasound feed below the transducer). The virtual rendering/representation of the needle and its linear trajectory consist of alternating cylinders or bands which mark distance away from the hub or base of the needle (e.g. inches). If the practitioner is using a 2 inch needle, then they know the needle tip is exactly two cylinders or bands away from the base or hub. As the practitioner advances the needle through the tissue with the virtual needle trajectory rendering/representation intersecting the target visible in the live ultrasound image projected below the transducer, the practitioner can estimate the distance from the needle tip to the target by counting the cylinders/bands between them.

In the medical and veterinary fields, needle length will vary based on a number of different criteria. The present invention does not require the needle to be a specific length or to somehow preset the virtual needle trajectory rendering (because it is independent and not based on/defined by the target location or needle length). The virtual needle trajectory rendering accurately represents length in increments (e.g. inches or cm) so that the needle guidance system allows the practitioner to know the needle tip depth and distance to any point beyond the tip.

If the practitioner wishes for the virtual needle trajectory rendering/representation to disappear or “turn off”, they simply spin the needle using their fingers so that the unique image/design/pattern is no longer detectable by the head-mounted display camera(s). Similarly, to make the virtual needle trajectory rendering reappear or “turn on”, they simply spin the needle within their fingers so that the unique image/design/pattern again becomes detectable by the head-mounted display camera(s). This is extremely useful because it allows the practitioner to see the physical needle in real scale in its exact physical location overlaid on the patient. This on/off functionality can also be achieved by practitioner voice commands

The proximity, alignment (X, Y, Z), and orientation (rotation) of the needle and transducer necessary to hit the intended target are determined by AR display device software and are used to successfully guide the needle to its target (in the tissue at some depth below the skin).

Software may be used to directly couple the AR/MR/VR head-mounted display to the ultrasound transducer and transmit the live ultrasound image feed from the transducer to the MR/AR/VR headset. Commands such as “take a picture”, “take a video”, “annotate the image”, “change depth”, “turn on Doppler”, etc. are voice-activated/hands-free and do not require the practitioner to turn away from the patient/procedure to press a button on the ultrasound machine.

Because the smaller true-to-size ultrasound feed displayed under the transducer is seen in the correct position (X, Y, Z) and orientation (rotation) of the transducer with respect to the practitioner or the AR display device camera(s), the correct spatial orientation of each pixel in the ultrasound feed is known and can be stored. In aggregate, these data can be used to construct and render 3D ultrasound images of tissue.

Plane alignment with needle and transducer—showing how the ultrasound device must first find and display the target and then, the practitioner can align the virtual needle guide/representation with the target before ever piercing the skin. As the needle pierces the skin and is then advanced through the tissue towards the target, the depth of the needle tip and distance to the target is known to the practitioner because of the alternating 1 inch bands of the virtual needle, which accurately indicate the length of the physical needle and its distance to the target.

Rotation of needle to make the virtual needle trajectory bands (depth markers) disappear/reappear—they only appear when the target image on the syringe/needle handle is visible to the camera(s) on the headset; by spinning the needle/rotating the target image out of the view of the camera(s) the MR needle trajectory disappears.

Display of live ultrasound feed on the AR/MR/VR headset affixed to a practitioner-specified portion of the field of view (top, bottom, left or right for example). This “large” ultrasound feed is analogous to a physical monitor—but is visible to the practitioner without them having to turn their head away from the patient. The live ultrasound content is identical to the smaller ultrasound feed described below.

A smaller live ultrasound feed is attached to the bottom of the transducer (via AR/MR, displayed as an overlay) to display tissue beneath skin surface (feed shown to scale of actual tissue). The position and orientation of the smaller ultrasound feed is attached to the bottom of the ultrasound transducer by tracking the image(s) on the transducer.

Free-hand mobility of transducer and needle/instrument which are independent of one another, and free practitioner movement in space with respect to the transducer and needle/instrument due to the head-mounted or stand-mounted AR display device camera(s). Practitioner does not need to turn their head since everything is in field of view via AR. Environment, such as room dimensions or even being outdoors does not constrain or prevent operation of invention.

The ultrasound feed is “live” (in real-time) from the transducer to the overlaid images. Indicia (e.g. highlighted box and/or halo) show when the needle trajectory intersects the plane of the ultrasound image. Software is used to couple the AR headset. to the ultrasound transducer and transmit the live feed from the transducer to the AR headset. Commands such as “take a picture”, “take a video”, “annotate the image”, “change depth”, are voice-activated and hands-free. Because the smaller true-to-size ultrasound feed displayed under the transducer is seen in the correct orientation of the transducer (with respect to the practitioner), the correct spatial orientation of each pixel in the images may be stored and used to render 3D images.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. Programming languages may include, but are not limited to: Ruby, JavaScript, Java, Python, Ruby, PHP, C, C++, C#, Objective-C, Go, Scala, Swift, Kotlin, OCaml, or the like. The program code may execute entirely on the computer, partly on the computer, as a stand-alone software package, partly on the computer, and partly on a remote computer or entirely on the remote computer or server. In the latter situation scenario, the remote computer may be connected to the computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method implemented on at least one computing device for facilitating a practitioner in guiding an insertion of a medical instrument into soft tissue of a patient; the method comprising:
receiving a first plurality of images from a medical imaging device;
receiving a second plurality of images from an augmented reality (AR) device interface, wherein the second plurality of images include fiducial-based information on both the medical instrument and the medical imaging device;
determining a predicted position of the medical instrument using fiducial-based localization with the fiducial-based information;
generating a third plurality of images includes illustrating the predicted position of the medical instrument within a first portion of the first plurality of images and within a first portion of the second plurality of images; and
transmitting the third plurality of images to the AR device interface.

2. The method of claim 1, wherein the third plurality of images are configured for providing a video overlay to an AR device associated with the AR device interface.

3. The method of claim 2, wherein the medical imaging device is an ultrasound imaging device.

4. The method of claim 3, wherein the ultrasound imaging device includes an ultrasound research interface (URI).

5. The method of claim 3, wherein the first plurality of images are B-mode images.

6. The method of claim 3, wherein the medical instrument may be a syringe and the syringe may include a needle.

7. The method of claim 6, wherein the fiducial-based information is provided by a first fiducial marker positioned on the medical instrument and a second fiducial market positioned on the medical imaging device.

8. The method of claim 7, wherein illustrating the predicted position of the medical instrument includes illustrating an extension of the needle.

9. The method of claim 8 further comprising, upon determining the extension of the needle is approximately within an imaging plane of a transducer of the ultrasound imaging device, providing an indicator within the third plurality of images.

10. The method of claim 9, wherein the indicator is a halo indicator positioned around an illustration of the predicted position and the illustration may be derived from a portion of the first plurality of images.

11. The method of claim 1, wherein the AR device interface is a radio frequency (RF) wireless interface.

12. The method of claim 11, wherein the AR device interface is at least one of a wireless personal area network (WPAN) interface and a wireless local area network (WLAN) interface.

13. The method of claim 1, wherein the AR device interface is an optical wireless interface.

14. The method of claim 1, wherein at AR device interface is provided by an AR display device.

15. The method of claim 14, wherein the AR display device has a field-of-view greater than 50 degrees.

16. The method of claim 14, wherein the AR display device has a resolution greater than 45 pixels per degree.

17. The method of claim 14 wherein the computing device and the AR device interface is embedded within the AR display device.

18. The method of claim 14, wherein the computing device is embedded within the medical imaging device.

19. A computing device for facilitating a practitioner in guiding an insertion of a medical instrument into soft tissue of a patient, the computing device comprising:
a memory; and
at least one processor configured for:
receiving a first plurality of images from a medical imaging device;
receiving a second plurality of images from an augmented reality (AR) device interface, wherein the second plurality of images include fiducial-based information on the medical instrument and the medical imaging device;
determining a predicted position of the medical instrument using fiducial-based localization with the fiducial-based information;
generating a third plurality of images illustrating the predicted position of the medical instrument within a first portion of the first plurality of images and within a first portion of the second plurality of images; and
transmitting the third plurality of images to the AR device interface.

20. A non-transitory computer-readable storage medium for facilitating a practitioner in guiding an insertion of a medical instrument into soft tissue of a patient, the non-transitory computer-readable storage medium storing instructions to be implemented on at least one computing device including at least one processor, the instructions when executed by the at least one processor cause the at least one computing device to perform a method, the method comprising:
receiving a first plurality of images from a medical imaging device;
receiving a second plurality of images from an augmented reality (AR) device interface, wherein the second plurality of images include fiducial-based information on the medical instrument and the medical imaging device;
determining a predicted position of the medical instrument using fiducial-based localization with the fiducial-based information;
generating a third plurality of images illustrating the predicted position of the medical instrument within a first portion of the first plurality of images and within a first portion of the second plurality of images; and
transmitting the third plurality of images to the AR device interface.
